(19) United States
(12) Patent Application Publication
(10) Pub. No.: US 2024/0335643 A1
(43) Pub. Date: Oct. 10, 2024

(54) SYSTEMS FOR WIRELESSLY DELIVERING ENERGY TO IMPLANTED MEDICAL DEVICES AND ASSOCIATED METHODS

(71) Applicant: Shifamed Holdings, LLC, Campbell, CA (US)

(72) Inventors: Peter Andriola, Castro Valley, CA (US); Brian Fahey, Menlo Park, CA (US); Mark Leo Kane, San Jose, CA (US)

(21) Appl. No.: 18/573,632

(22) PCT Filed: Jun. 30, 2022
(86) PCT No.: PCT/US2022/035764

§ 371 Date: Dec. 22, 2023

Related U.S. Application Data
(60) Provisional application No. 63/217,081, filed on Jun. 30, 2021.

(51) Int. Cl. A61M 27/00

(52) U.S. Cl. CPC A61M 27/002; A61M 2205/04; A61M 2205/3303; A61M 2205/3327; A61M 2205/3331; A61M 2205/3584; A61M 2205/3592; A61M 2205/52; A61M 2205/8206; A61M 2205/8243; A61M 2210/125

(57) ABSTRACT

A system for delivering energy to implanted devices using electromagnetic wireless charging and associated systems and methods are disclosed herein. In some embodiments, the system includes an energy transmission device and an implanted device. The energy transmission device can include multiple transmission coils, while the implanted device can include one or more receiving coils and one or more chargeable energy storage components. The transmission coils on the energy transmission device can each be energized to generate an electromagnetic field. When the implanted device is positioned within range of the energy transmission device, the receiving coils in the implanted device interact with the electromagnetic fields to generate a current. The current is used to charge the energy storage components.

### CROSS-REFERENCE TO RELATED APPLICATION(S)

This application claims the benefit of U.S. Provisional Patent Application No. 63/217,081, filed Jun. 30, 2021, and incorporated by reference herein in its entirety.

### TECHNICAL FIELD

The present technology generally relates to wireless energy transfer systems for implantable medical devices and, in particular, to wireless energy transfer systems for implantable devices for treating cardiovascular conditions and associated methods and devices.

### BACKGROUND

Wireless charging has become a popular mode for providing power to energy storage components in many devices, including certain medical implant devices that consume higher power than traditional implanted devices (e.g., cardiac rhythm management devices, etc.). These implanted devices have significantly limited operational lifetimes when powered only by a primary (e.g., non-rechargeable) battery. Additionally, implanted devices that are delivered using minimally-invasive techniques may have strict, or practically-limited, maximum sizes for energy storage components in order to be compatible with minimally invasive delivery systems and/or the target anatomy. The size limitations in turn limit the available energy storage capacity of a primary battery, further limiting the operational lifetimes of such devices when powered only by a primary battery of a suitable size to be compatible with the desired delivery system. Implanted devices that incorporate a rechargeable energy storage component sometimes require another invasive procedure to recharge the rechargeable energy storage component. Alternatively, wireless charging of the rechargeable energy storage component can help prolong the operational lifetime of the implanted devices without requiring additional invasive procedures. However, conventional wireless charging schemes typically require strict alignment between the charging device and the receiving device, and often require bulky and uncomfortable equipment to be worn by the patient. Such recharging processes can be inconvenient, time-consuming, and require strict patient compliance, which is challenging to maintain over time. As a result, patients often (willingly or unwillingly) fail to charge the energy storage components of their implanted device, and therefore lose the beneficial medical effects of the implanted device once the device exhausts its available stored on-board energy.

### BRIEF DESCRIPTION OF THE DRAWINGS

FIG. 1 is a partially schematic illustration of a system for wirelessly delivering energy to and communicating with an implanted device configured in accordance with select embodiments of the present technology.

FIG. 2A is a schematic illustration of a system for wirelessly delivering energy to an implanted interatrial shunting element configured in accordance with some embodiments of the present technology.

FIGS. 2B and 2C are schematic illustrations of the system of FIG. 2A for wirelessly delivering energy to an implanted device in accordance with some embodiments of the present technology.

FIG. 3 is a partially schematic top view of an energy transmission device for wirelessly delivering energy to an implanted device in accordance with some embodiments of the present technology.

FIGS. 4A-4D are schematic diagrams of the magnetic fields produced by an energy transfer device in accordance with some embodiments of the present technology.

FIG. 4E is a vector diagram of the magnetic field lines proximate to an implanted device positioned above the energy transfer device in accordance with some embodiments of the present technology.

FIGS. 5A and 5B are partially schematic top views of a printed circuit board configured in accordance with embodiments of the present technology.

FIGS. 5C and 5D are partially schematic cross-sectional views of the printed circuit board of FIG. 5A in accordance with some embodiments of the present technology.

FIGS. 6A and 6B are schematic illustrations of a system for wirelessly delivering energy of the type illustrated in FIG. 2A configured in accordance with further embodiments of the present technology.

FIG. 7 is a block diagram of a system for wirelessly delivering energy to an implanted device configured in accordance with further embodiments of the present technology.

The drawings have not necessarily been drawn to scale. Similarly, some components and/or operations can be separated into different blocks or combined into a single block for the purpose of discussion of some of the implementations of the present technology. Moreover, while the technology is amenable to various modifications and alternative forms, specific implementations have been shown by way of example in the drawings and are described in detail below. The intention, however, is not to limit the technology to the particular implementations described.

### DETAILED DESCRIPTION

### A. Overview

The present technology is directed to wireless energy transfer systems for implantable medical devices, such as implantable interatrial shunts or other suitable implantable devices. In some embodiments, a system for delivering energy to implanted devices comprises an energy transmission device and an implanted device that can receive energy from the energy transmission device. The energy transmission device includes multiple transmission coils, while the implanted device can include one or more receiving coils and one or more chargeable energy storage components. The transmission coils on the energy transmission device can each be energized to generate an electromagnetic field having a selected frequency (e.g., about 6.78 MHz). Further, in some embodiments, the coils can be offset in a longitudinal plane and the energy transmission device can energize individual transmission coils sequentially. As the transmission coils are sequentially energized, the transmission coils generate multiple electromagnetic fields that are offset from each other according to the offset of the transmission coils. When the implanted device is positioned within range of the energy transmission device, the receiving coil(s) in the implanted device are subject to varying magnitudes and orientations of the electromagnetic fields and accordingly can generate one or more currents that can charge the energy storage components. As a result, the inventors have discovered that the receiving coil(s) can be at least partially aligned with at least one of the magnetic fields when the patient is proximate to the energy transmission device for even a relatively short period of time, thereby ensuring that at least some energy is delivered to the implanted device without significant effort or complicated protocols required by the patient to carefully align the energy transmission device with the implanted device.

As explained in further detail below, the present technology is expected to simplify wireless charging of implanted devices and/or to reduce inconvenience and unpleasantness associated with the charging of implanted medical devices. For example, conventional wireless charging systems typically require a relatively bulky external charging device to be placed in close proximity with an implanted device of a patient during an energy transfer process. The external device can include a charging coil and be held in place by a belt, band, or other garment worn by the patient during the charging session. Such charging devices, however, are inconveniently large, can become uncomfortably warm during energy transfer, and require very strict alignment between the charging coil and a receiving coil. Consequently, successful recharging of the implanted device can require the patient to follow cumbersome, strict procedures to ensure the success of the energy transfer process. As a result, patients can neglect, avoid, or err in recharging their implanted devices, thereby losing the intended clinical benefits of the device. In contrast with such conventional systems, and as explained in further detail below; the present wireless charging technology is expected to minimize the requirements for patient compliance with regard to the charging of implanted devices and help significantly improve patient compliance.

Another problem associated with many conventional wireless charging system concepts is that such system concepts require significant energy for operation. For example, some conventional wireless charging systems include using either a plurality of large magnetic transmitting coils or a plurality of ultra-high frequency (UFH) transmitters. The large coils create a magnetic field throughout an entire room or house to charge a device located anywhere in the room or house. However, the magnetic field approach is impractical for use because very high field amplitudes need to be produced at the source coils in order to generate adequate field strength at the implanted device, which requires substantial electrical power/energy consumption to operate. The UHF approach is limited by safety and regulatory concerns such as limits to the tissue specific absorption rate (SAR), which undermines the UHF approach as a suitable solution except in the lowest power applications. As also explained in detail below, embodiments of the present technology are also expected to reduce the power requirements associated with the charging of implanted devices using systems that remove the need for active patient participation.

In various embodiments, the energy storage component(s) of the implanted devices include secondary (e.g., rechargeable) battery cells, supercapacitors, and/or any other suitable storage components. Secondary battery cells are a proven technology for energy storage components. However, the secondary battery cells must be charged relatively slowly (e.g., over a period of about 10 hours) and, similarly, must be discharged relatively slowly. This limits their practicality for a number of devices that require rapid charging and/or rapid discharge of energy. Further, the secondary battery cells have a relatively limited number of charge/discharge cycles in their functional operating life (e.g., about 500 cycles), at the end of which the patient must undergo another procedure to replace the battery in order to maintain the benefits of the implanted device. A supercapacitor, in contrast, can be charged and discharged much more quickly than a secondary battery cell. For example, in various embodiments, a supercapacitor for use in the implanted device can be charged in about 1 second, about 10 seconds, about 1 minute, about 10 minutes, about 20 minutes, or another suitably short period of time. Further, supercapacitors can have a much higher cycle life than secondary battery cells (e.g., more than about 100,000 cycles on average), allowing the patient to avoid and/or reduce the number of procedures necessary to maintain the benefits of the implanted device. However, the use of a supercapacitor requires systems and methods that are adapted to supercapacitor technology.

In some embodiments, the energy transmission device (and/or charging system in general) configured in accordance with the present technology includes features that minimize the requirement for patient compliance with a wireless charging regimen. As explained in further detail below; these features enable autonomous operation (e.g., operation that does not require dedicated actions from the patient, such as careful alignment between the energy transmission device and the implanted device) and/or features that enable charging without the need for a body-worn transmitter. Indeed, in some embodiments, the operation of the energy transmission device and the interface between the energy transmission device and the implanted device is entirely automatic, requiring no patient input or action. In some embodiments, the energy transmission device includes transmission coils that are relatively large compared to conventional body-worn coils. The large coils allow the charging system to generate a sufficient magnetic field to deliver adequate energy to charge and/or power the implanted device despite a larger (relative to conventional systems) physical separation from the implanted device and/or any attenuating media between the charging system and the implanted device. In some embodiments, the charging system can be located such that the patient will regularly (i.e., in the routine course of their daily life) be proximate to the charging coils. For example, the energy transmission device can be integrated into and/or positioned adjacent to an object such as a patient's bed (e.g., a mat placed under a mattress), a patient's chair or couch (e.g., in a cushion, blanket, or cover), an office desk, a rug or carpet, or another suitable object that the patient frequently interacts with.

In some embodiments, the charging system includes features that minimize the likelihood that a patient (or a third party) purposefully/accidentally disconnects or otherwise disables the charging system. For example, the charging system can limit or eliminate the visible presence of one or more components of the charging system and/or reduce the energy consumption (and thereby the financial operating cost) of one or more components of the charging system. In some embodiments, the consequence of limiting the visible presence of the system is an additional distance between the charging system and the implant and/or the presence of additional attenuating media between the charging system and the implant. As a result, the energy consumption of the charging system can go up, further requiring features that minimize or reduce the energy consumption.

In addition to energy consumption, automation, and attenuation concerns, patient exposure and related regulatory concerns must also be considered in the design of charging systems as described herein. For example, radiofrequency magnetic fields induce the presence of companion radiofrequency electric fields. Human soft tissue is an electrically conductive medium, so the induced electric fields in turn create an electric current density in the tissue. To limit patient exposure in compliance with regulatory standards and/or other best practices, embodiments of the present technology are configured to minimize the magnitude of induced electric fields. For example, as discussed in more detail below, the charging system can use a sufficiently low radiofrequency to avoid undesirable exposure effects.

Additional details on various features of the charging system introduced above are described with respect to the figures below. Although primarily discussed herein as a system for delivering energy to an interatrial shunting element, one of skill in the art will understand that the scope of the present technology is not so limited. For example, the system can also wirelessly deliver energy to one or more alternative, or additional, implanted devices. For ease of reference, the components of the system for delivering energy to implanted medical devices are sometimes described herein with reference to top and bottom, upper and lower, upwards and downwards, and/or longitudinal planes, x-y plane, vertical, or z-direction relative to the spatial orientation of the embodiments shown in the figures. It is to be understood, however, that the components of the system can be moved to, and used in, different spatial orientations without changing the structure and/or function of the disclosed embodiments of the present technology.

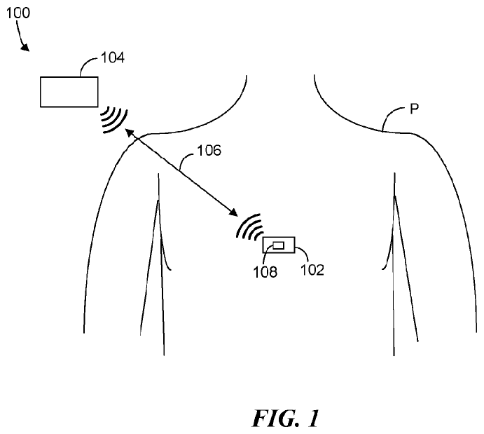

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-7.

Reference throughout this specification to relative terms such as, for example, “generally,” “approximately,” and “about” are used herein to mean the stated value plus or minus 10%.

Throughout this specification, “power” and “energy” are used somewhat interchangeably. One of ordinary skill in the art will appreciate, however, that power and energy are not always equivalent. For example, a battery can viewed as both an “energy” storage or “power” storage component. However, “power” may also refer to a rate at which stored energy is discharged. Thus, one of ordinary skill in the art will understand that the terms “power” and “energy” may have a same or different meaning in various places throughout this specification based the context and/or manner in which these terms are used.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a LA of a heart) and a second region (e.g., a RA or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system or other system. For example, any of the shunts described herein, including those referred to as “interatrial,” may be nevertheless used and/or modified to shunt between the LA and the coronary sinus, or between the right pulmonary vein and the superior vena cava. Moreover, while the disclosure herein primarily describes medical devices for shunting blood in the heart, the present technology can be readily adapted for medical devices used to shunt other fluids—for example, devices used for aqueous shunting or cerebrospinal fluid shunting. The present technology may also be adapted to a variety of implanted medical devices in addition to shunts.

The headings provided herein are for convenience only and do not interpret the scope or meaning of the claimed present technology.

### B. Systems for Wirelessly Delivering Energy to and Communicating with an Implanted Device and Associated Methods

FIG. 1 is a partially schematic illustration of a system 100 for non-invasively providing energy to and communicating with an implanted device 102 in a patient P in accordance with select embodiments of the present technology. As illustrated in FIG. 1, the system 100 can include a first device 102 (also referred to as “the implanted device 102”) implanted or configured to be implanted in the patient P and a second device 104 (also referred to as “the external device 104”) external to the patient P. The implanted device 102 includes an internal component 108 that can transmit electromagnetic energy to and/or toward the external device 104 along a transmission path 106. The internal component 108 can transmit data using any suitable type of wired or wireless communication method, including electromagnetic, ultrasound, or radiofrequency (e.g., WiFi, Bluetooth (such as BLE 5.0), MEDRadio, ZigBee, sub-GHz). Alternatively, or additionally, the external device 104 can transmit electromagnetic energy to and/or toward the internal component 108 of the implanted device 102 along the transmission path 106. For example, the external device 104 can transmit magnetic waves towards the implanted device 102 to wirelessly deliver energy to the implanted device 102. That is, the internal component 108 can be configured as both a transmitter and a receiver. In some embodiments, the implanted device 102 includes multiple internal components 108. For example, a first internal component 108 can be configured to transmit communications towards the external device 104 while a second internal component 108 can be configured to receive energy from the external device 104. Similarly, in various embodiments, the external device 104 can be configured as a transmitter and/or a receiver. In some embodiments, the system 100 can include multiple external devices 104. For example, a first external device 104 can transmit energy towards the implanted device 102 while a second external device 104 can send and/or receive communications from the implanted device 102.

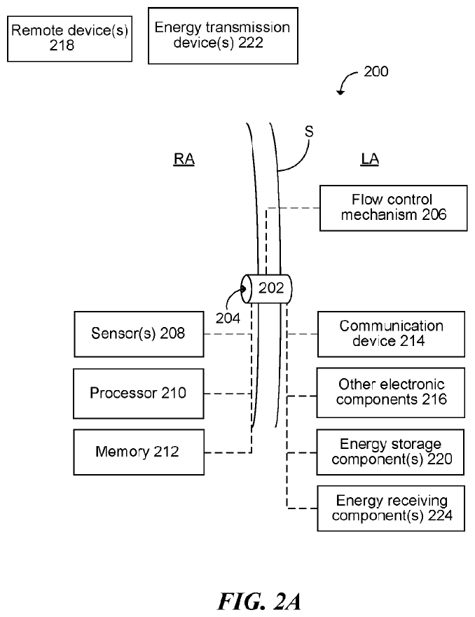

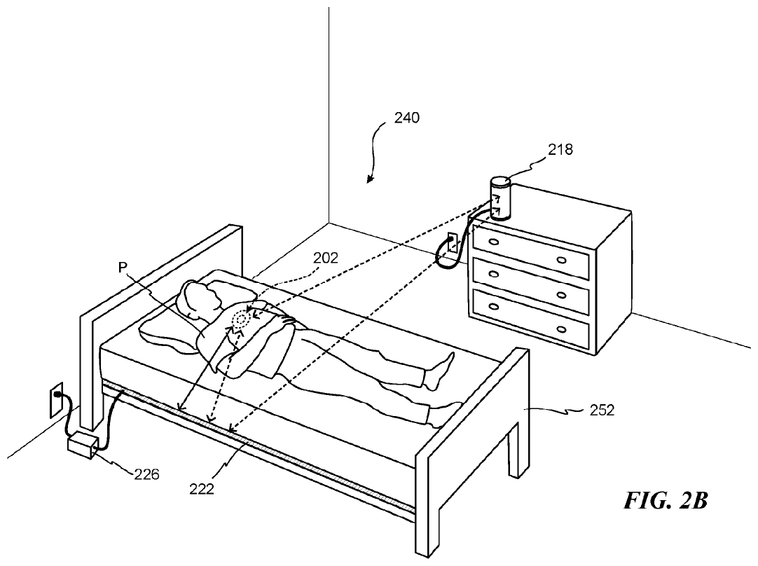

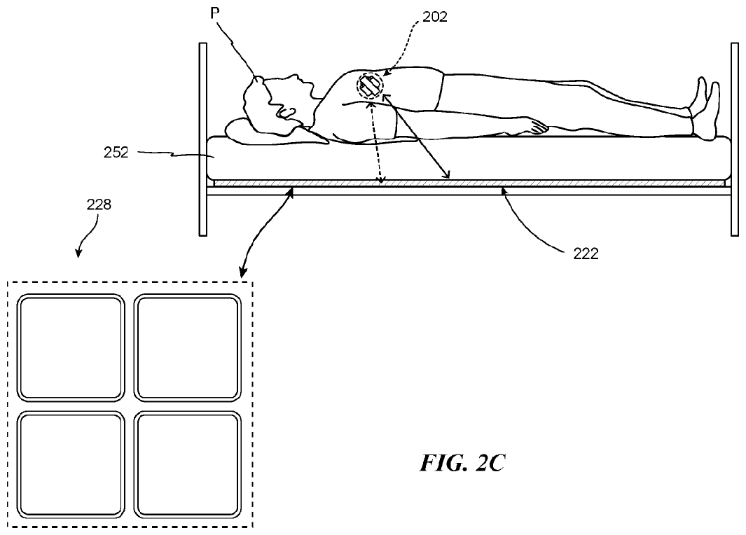

Additional details regarding representative embodiments of the present technology are described below with reference to FIGS. 2A-7. Although described primarily herein as a system delivering energy to an interatrial shunting element, one of skill in the art will understand that the scope of the present technology is not so limited. For example, the system described herein can non-invasively deliver energy to any other suitable implanted device. Non-limiting examples of implanted devices that can require power include a cardiac pacemaker, interatrial shunt, artificial heart valve, cardioverter-defibrillator, coronary stent, cochlear implant, tympanostomy shunt, neurostimulator, and artificial urinary sphincters. Further, although the external energy delivery device is described primarily herein as positioned underneath a mattress, one of skill in the art will understand that the scope of the present technology is not so limited. For example, the external energy delivery device can be positioned beneath a rug, positioned beneath a carpet, incorporated into a desk, incorporated into a table, and/or positioned/incorporated into any other suitable location.

FIG. 2A is a schematic illustration of a system 200 for wirelessly delivering energy to an implanted interatrial shunting element 202 (also referred to as “shunting element 202”) configured in accordance with embodiments of the present technology. The system 200 includes the shunting element 202 defining a lumen 204 therethrough. When implanted in the septal wall S, the system 200 can fluidly connect the left atrium LA and the right atrium RA via the lumen 204. The system 200 can also include various electronic components carried by, operably coupled to, and/or otherwise associated with the shunting element 202, such as a flow control mechanism 206, one or more sensors 208, a processor 210, memory 212, a communication device 214, and/or other electronic components 216. Any or all of these electronic components may be configured as active electronic components (e.g., require power from an energy source to operate). Accordingly, the shunting element 202 can include and/or be operably coupled to a plurality of energy sources, such as one or more energy storage components 220 operably coupled to (and/or incorporated into) the shunting element 202 within the patient's body and/or one or more energy transmission device(s) 222 external to the patient's body. As further illustrated in FIG. 2A, the shunting element 202 can also include one or more energy receiving component(s) 224 operably coupled to (and/or incorporated into) the shunting element 202 and/or components thereof. As discussed in more detail below, the energy receiving component(s) 224 are configured to wirelessly receive energy from the energy transmission device(s) 222, thereby facilitating non-invasive energy transfer to components of the shunting element 202. The electronic components can be configured to draw power from the energy storage component(s) 220 and/or the energy receiving component(s) 224 to perform various operations, such as the functions described below.

The processor 210 (e.g., a microprocessor, microcontroller, FPGA, ASIC, electronic control hardware, etc.) can be configured to perform various operations in accordance with corresponding instructions stored in the memory 212. For example, the processor 210 can be configured to receive data from the sensor(s) 208 and, optionally, store the data from the sensor(s) 208 in the memory 212. As another example, the processor 210 can be configured to calculate a pressure differential between the left atrium LA and the right atrium RA based on sensor data and/or other relevant calculations, and/or store the calculated pressure differential in the memory 212. In some embodiments, the processor 210 is operably coupled to the flow control mechanism 206 to control adjustments to the shunting element 202 and/or lumen 204, e.g., based on the parameters measured by the sensor(s) 208, the pressure differential calculated by the processor 210, input from a care provider or user, and/or other relevant data received by the processor 210 and/or stored in the memory 212. For example, if the calculated pressure differential falls outside of a predetermined range, the processor 210 can direct the flow control mechanism 206 to adjust the amount of blood flow through the shunting element 202. In some embodiments, the sensor(s) 208, processor 210, and the flow control mechanism 206 operate in a closed-loop system to adjust the shunting element 202.

In some embodiments, the processor 210 is configured to transmit data (e.g., sensor data, calculated pressure differential, data related to the health of the energy storage component(s) 220, data related to the charging cycles through the energy receiving component(s) 224, etc.) via the communication device 214 to one or more remote device(s) 218 located outside the patient's body (e.g., a controller mobile device, computing device, Internet of Things (IoT) networked device, virtual AI assistant, stand-alone data reader/interrogator, etc.). For example, the communication device 214 can be configured to transmit data from the sensor(s) 208 to a virtual AI assistant, which in turn uploads the data to an external hub or reader (e.g., for notification purposes, for processing and/or analysis, etc.). The communication device 214 can use any suitable type of wired or wireless communication method, including electromagnetic, ultrasound, or radiofrequency (e.g., WiFi, Bluetooth (such as BLE 5.0), MEDRadio, ZigBee, sub-GHz (e.g., in the range of about 850 MHz to about 950 MHz)). In some embodiments, the communication device 214 can include multiple devices and/or implement multiple different communication modalities, e.g., to provide improved operational flexibility and reduce the power and/or energy requirements of the system 200. In some embodiments, the processor 210 is configured to transmit data via the communication device 214 in response to receiving power from the energy receiving component(s) 224.

The processor 210 can also be configured to receive data (e.g., control signals) from the remote device(s) 218 via the communication device 214. The control signals can indicate that one or more components of the system 200 are to be operated. For example, the control signals can direct the sensor(s) 208 to measure one or more parameters and direct the processor 210 to store the sensor data in the memory 212 and/or transmit the sensor data to the remote device(s) 218. As another example, the control signal can direct the flow control mechanism 206 to adjust the shunting element 202 and/or lumen 204. In some embodiments, for example, a physician communicates a desired parameter for the shunting element 202 to the remote device(s) 218 (directly or through a network connection to the remote device(s) 218), and the remote device(s) 218 communicates the adjustment to the shunting element 202 (via the communication device 214 and processor 210).

Additional details on the functioning of the active electronic components of the shunting element 202 (e.g., sensor(s) 208, processor 210, memory 212, communication device 214, other electronic components 216, etc.) and/or the construction of the shunting element 202 according to some embodiments of the technology can be found in International Application No. PCT/US2021/028931, the disclosure of which is incorporated herein by reference in its entirety.

As discussed above, the active electronic components require power from at least one energy source to operate. Accordingly, the shunting element 202 can include and/or be operably coupled to a plurality of energy sources for powering operation of the active electronic components. The energy sources can include any suitable combination of chargeable and non-rechargeable energy sources, and can include sources located external to the patient's body as well as sources located within the patient's body. In the illustrated embodiment, the energy sources include the energy storage component(s) 220 and energy receiving component(s) 224 (both internal to the patient's body), as well as the energy transmission device(s) 222 (external to the patient's body). The energy sources can each be associated with a respective set of power output characteristics correlating to an amount of power that can be delivered by the energy source. The power output characteristics can include, for example, a maximum or peak power output, a minimum power output, and/or an average power output. In some embodiments, some or all of the energy sources can have different power output characteristics, as described in greater detail below. Alternatively, or in combination, each of the energy sources can also be associated with energy storage characteristics, which in some embodiments may differ between some or all of the energy sources.

The energy storage component(s) 220 can include a primary battery (i.e., a non-rechargeable battery), a secondary battery (i.e., a rechargeable battery), a capacitor, a supercapacitor, and/or other suitable elements that can store and/or provide energy to the system. In some embodiments, the system 200 includes at least two different types of energy storage component(s) 220. For example, the energy storage component(s) 220 can include two or more of a primary battery, a secondary battery, a supercapacitor, or a capacitor. In one embodiment, for example, the energy storage component(s) 220 includes a secondary battery and a supercapacitor component.

In some embodiments, the energy storage component(s) 220 are each associated with a respective set of power output characteristics (e.g., a maximum or peak power output, a minimum power output, an average power output, etc.). In some embodiments, the energy storage component(s) 220 are each associated with a respective set of power receiving characteristics (e.g., a charge time, a cycle life (i.e., the number of times the component can be charged), a minimum power input to charge, etc.). The energy storage component(s) 220 can also be associated with other characteristics relevant to power management, such as an energy density (e.g., a volumetric energy density), an energy storage capacity, a power density (e.g., a volumetric power density), and/or a self-discharge time (i.e., how long the component can retain energy). In some embodiments, some or all of the energy storage component(s) 220 can have different characteristics. For example, a primary battery can have an energy density that is different from (e.g., greater than) an energy density of a secondary battery, which can have an energy density that is different from (e.g., greater than) an energy density of a supercapacitor, which can have an energy density that is different from (e.g., greater than) an energy density of a capacitor. As another example, a primary or non-rechargeable battery can have a power density that is different from (e.g., less than) a power density of a secondary or rechargeable battery, which can have a power density that is different from (e.g., less than) a power density of a supercapacitor, which can have a power density that is different from (e.g., less than) a power density of a capacitor.

In some embodiments, the energy storage component(s) 220 are configured for delivery into the patient's heart via percutaneous and/or catheter delivery techniques. As a result, the size (e.g., volume) of the energy storage component(s) 220 can be limited based on the size of the catheter used. Additionally, or alternatively, size limitations can be associated with the anatomical placement location of the energy storage component(s) 220. For example, energy storage component(s) 220) can be intended for placement on the atrial septal wall of a patient's heart. In this location, the length of the energy storage component(s) 220 can be limited to avoid approaching sensitive patient anatomy (e.g., heart valves, pulmonary vessels, etc.). Any of these size constraints can limit the energy storage capacity of an individual energy storage component 220. The use of the energy transmission device(s) 222 external to the patient's body is expected to ameliorate the limitations imposed by the size constraints by supplementing the energy storage component(s) 220 with a consistent source of external energy. Further, the use of energy storage component(s) 220 that can quickly charge (e.g., supercapacitors) is also expected to improve the efficacy of the system 200 using the energy transmission device(s) 222.

The energy transmission device(s) 222 include an individual device or system that is capable of wirelessly transmitting energy to the shunting element 202 and/or any component of the shunting element 202 (e.g., an inductive wireless charging device). For example, as described in more detail below with respect to FIGS. 2C-4E, each of the energy transmission device(s) 222 can include one or more transmission coils that generate an electromagnetic field when the coil is energized (e.g., by a current passed through the coil). The magnetic field can interact with one or more receiving coils comprising at least a portion of the energy receiving component(s) 224 to generate a current therein, thereby transferring energy to the energy receiving component(s) 224.

If the energy transmission device(s) 222 (and the transmission coil therein) is located remote from a patient (e.g., is not worn on or very close to the patient's body), the electromagnetic field strength in the vicinity of the transmission coil must be large to compensate for attenuation effects that the transmitted magnetic field will experience due to the distance from the energy transmission device 222 to the energy receiving component(s) 224. Accordingly, a transmission coil associated with a remotely-located energy transmission device 222 must produce a larger overall electromagnetic field than that produced by a body worn system. In turn, the requirement to produce a larger electromagnetic field requires that the voltage applied across a remotely-located transmission coil must be relatively higher (e.g., 500-2500 V_{RMS}) than the voltage applied to a body-worn coil (e.g., 10-100 V_{RMS}). The larger magnitude of the applied voltage leads directly to a larger magnitude in electrical currents in the transmission coil, leading to increasing losses from electrical resistance in the coils and increasing the cost of continuous operation. Accordingly, in various embodiments, the energy transmission device(s) 222 can include features that minimize the operating power required by the energy transmission device(s) 222, the amount of time the energy transmission device(s) 222 are operated to successfully charge the energy storage component(s) 220, and/or the standby power associated with the energy transmission device(s) 222. For example, one significant source of operating power consumption can be resistive heating in the transmission coil's conductors. Resistive heating in a metallic structure can be a function of the magnitude of current flowing through the structure, the dimensions of the structure, and the electrical resistance of the structure. Accordingly, in some embodiments, the energy transmission device(s) 222 reduce their operating power by maximizing the cross-sectional area of the current-carrying part of the transmission coils and/or including high conductivity conductor materials to reduce the losses due to resistance.

Alternatively, or additionally, the energy transmission device(s) 222 can include one or more sensors (not shown) that detect the presence of the patient and/or that the energy receiving component(s) 224 are within a predetermined range of the energy transmission device(s) 222. For example, in some embodiments, one or more appropriate components (e.g., a microcontroller controlled by firmware) that are integrated into or otherwise functionally coupled to the energy transmission device(s) 222 periodically send out test signals to detect the presence of the shunting element 202 within a specified region. If the shunting element 202 is detected within the specified region, the transmission coils of the energy transmission device(s) 222 can be energized to transmit energy toward the shunting element 202. In some embodiments in which the energy transmission device(s) 222 are integrated into or otherwise proximate to a surface (e.g., a rug, seat cushion, mattress, etc.) that the patient is expected to interface with regularly, the energy transmission device(s) 222 include pressure sensors to sense pressure applied to the surface. When the pressure sensor detects a pressure above a predetermined threshold (e.g., indicating that the patient may be proximate to the energy transmission device(s) 222), the transmission coils of the energy transmission device(s) 222 can be energized to transmit energy toward the shunting element 202. In some embodiments, a combined pressure sensor/test signal system can be implemented (e.g., test signals are only sent out if a pressure sensor detects weight on the surface). In some embodiments, the energy transmission device(s) 222 operate only when the shunting element 202 is within a predetermined range to reduce the operating time of the energy transmission device(s) 222. During other periods, the energy transmission device(s) 222 can operate in a low-power standby mode and/or can be completely powered off.

As discussed above, in some embodiments, the energy transmission device(s) 222 are located remote from the patient in a position that the patient frequently (e.g., hourly, daily, weekly) becomes proximate to as part of their normal routine. In various embodiments, for example, the energy transmission device(s) 222 are integrated into an object such as a patient's bed (e.g., a mat placed under a mattress), a patient's chair or couch (e.g., in a cushion, blanket, cover, or a mat placed under a cushion or chair), a rug or carpet, a desk, or any other suitable object. In some such embodiments, the remote location introduces a significant amount of attenuating media (e.g., a thick mattress, patient's body, the air, and/or other intervening media) between the transmission coil in the energy transmission device(s) 222 and the patient. Accordingly, in such embodiments, the frequency of the current used to excite a transmission coil (and thereby create an electromagnetic field) is selected at least in part to minimize losses due to absorption in the attenuating media while delivering ample energy to the energy receiving component(s) 224. For example, the electromagnetic field can be generated at a low radio frequency that experiences less absorption in the attenuating media, for example between 0.1 MHz and 20 MHz. In various such embodiments, the electromagnetic field can be generated at a transmission frequency in the range of about 20 kHz to about 20 MHz (e.g., about 6.78 MHz, about 13.56 MHz).

The system 200 can also include features that account for the lack of precise alignment between the transmission coil in the energy transmission device(s) 222 and the receiving coil in the energy receiving component(s) 224. For example, as discussed in more detail below with respect to FIGS. 2C-4E, the energy transmission device(s) 222 can include a plurality of transmission coils at least partially offset from each other in a longitudinal plane. In some embodiments, the plurality of transmission coils can be at least partially offset from each other in multiple planes. During use, the transmission coils can be energized together or separately, and/or the selection of which transmission coil(s) are energized can vary over time. By sequentially energizing the transmission coils (or combinations thereof), the shape, magnitude, and/or directionality of the transmitted electromagnetic field can be varied, thereby changing the relative orientation between the transmitted electromagnetic field and receiving coil(s). As a result, a wide array of possible patient positions can be at least partially aligned with the electromagnetic field from the energy transmission device(s) 222 to receive the energy transfer. The wide array of possible positions allows the system 200 to operate without the need for patient interaction (e.g., for precise coil alignment).

In addition to attenuation and alignment concerns, patient exposure and related regulatory concerns must also be considered in the system 200 as described herein. For example, radiofrequency magnetic fields induce the presence of companion radiofrequency electric fields. Human soft tissue is an electrically conductive medium. Accordingly, the magnetic fields transmitted by energy transmission device(s) 222 can induce electric fields in the tissue. The induced electric fields can create an electric current density in the tissue. To limit patient exposure in compliance with regulatory standards and/or other best practices, some embodiments of the present technology are configured to minimize the magnitude of induced electric fields. For example, the transmission and receiving coil(s) can be separated by a distance D. The wavelength of the magnetic field can be represented by λ, E is the magnitude of the electric field in volts per meter (V/m), and H is the magnitude of the magnetic field in Amperes per meter (A/m). If D>(λ/2π), the ratio E/H can be relatively high (e.g., >350 Ohms), which results in relatively high-magnitude electrical fields (e.g., 1,000 V/m). At longer wavelengths, for example where D<0.05*λ/2π, the ratio E/H is relatively lower, (e.g., 20 Ohms), which results in lower-magnitude electrical fields (e.g., ˜50 V/m). The volumetric power dissipation in tissue is proportional to E². Accordingly, embodiments that utilize longer wavelengths reduce the power dissipated in the tissue. In some embodiments, the energy transmission device(s) 222 can use a transmission frequency of about 6.8 MHz (e.g., of about 6.78 MHz) to ensure D is less than (or close to) 0.05*λ/2π even when the separation between the coils is about 35 cm (e.g., when the energy transmission device(s) 222 is positioned beneath a mattress). In various embodiments, depending on the separation distance, a suitable radiofrequency can be a higher frequency than 6.78 MHz (e.g., 13.56 MHz, 10 MHz, or 20 MHz) and/or a lower frequency than 6.78 MHz (e.g., 5 MHz, 1 MHz, 100 kHz, 90 KHz, 50 kHz, or 20 kHz).

As discussed above, the system 200 includes one or more energy receiving component(s) 224 operably coupled to the shunting element 202 and configured to receive energy from the energy transmission device(s) 222. The energy receiving component(s) 224 can be (or can include) one or more metallic coils adapted to receive electromagnetic energy transmitted from the energy transmission device(s) 222. In various embodiments, the energy receiving component(s) 224 can be made of, or include, copper, silver, gold, aluminum, stainless steel, nitinol, another suitable material, and/or suitable combinations of these materials. The receiving coil(s) in the energy receiving component(s) 224 interact with the transmitted magnetic field, thereby generating a corresponding current through the receiving coil(s). The current can then be used to power and/or charge various components of the shunting element 202. Optionally, the energy receiving component(s) 224 can also be configured to transmit energy and/or signals to the remote device(s) 218. In such embodiments, the energy receiving component(s) 224 can also operate as part of the communication device 214 for the shunting element 202. In other embodiments the energy receiving component(s) 224 can be different from the communication device 214.

In some embodiments, energy transmitted from the energy transmission device(s) 222 and received by the energy receiving component(s) 224 is used to directly power operation of one or more components of the system 200. Alternatively, or in combination, energy transmitted from the energy transmission device(s) 222 and received by the energy receiving component(s) 224 can be stored in the energy storage component(s) 220. For example, the energy transmission device(s) 222 can be used to wirelessly recharge a rechargeable battery, a supercapacitor, or a capacitor. Subsequently, the energy stored in the energy storage component(s) 220 can be used to power operation of one or more active electronic components of the system 200. For example, the flow control mechanism 206 can be adjustable using energy stored in the energy storage component(s) 220. Accordingly, the energy storage component(s) 220) can store energy for a period of time (e.g., hours, days, months, etc.) and, when directed by the external device(s) 218 and/or the processor 210, the energy storage component(s) 220 can release stored energy to one or more components of the shunting element 202.

In some embodiments, the energy receiving component(s) 224 generate a current that is used to charge a secondary battery. However, in some such embodiments, the relatively long required charging times of a secondary battery can limit the utility of the system 200 as described above that autonomously solves challenges associated with transmission and receive coil alignment. For example, for any given patient position, many of the transmitted electromagnetic fields created by the energy transmission device(s) 222 will not align well enough with a receiving coil to produce meaningful charging. Time spent transmitting these poorly aligned fields is thus “wasted,” and lowers the cumulative effective charging time for the implant to a fraction of the total patient-system interface time. That is, in embodiments that sequentially and frequently shift the electromagnetic field orientation (e.g., by frequently shifting which transmission coils are energized), the effective charging time can be shorter compared to carefully and statically-aligned devices. Accordingly, in some embodiments, the energy transmission device(s) 222 as described above are particularly useful when the energy storage component(s) 220 include a supercapacitor, which can be charged relatively quickly. In some such embodiments, the energy storage component(s) 220 include both a supercapacitor and a secondary battery. During one mode of operation for such embodiments, the system 200 can be configured to initially charge the supercapacitor. Once the supercapacitor is completely charged, any “excess” interface time between the energy receiving component(s) 224 and the energy transmission device(s) 222 can be utilized to send at least some energy to a secondary battery. Additionally, or alternatively, the supercapacitor can be configured to charge the secondary battery during between periods of interface time between the energy receiving component(s) 224 and the energy transmission device(s) 222.

Further, as described above, the active electronic components of the system 200 can have different power consumption characteristics, while the energy sources associated with the system 200 can have different power output characteristics. To improve efficiency and performance, the system 200 can implement a power management scheme to optimize or otherwise improve the manner in which the active electronic components are powered by the energy sources. Accordingly, rather than allowing power allocation to be passively dictated by the inherent characteristics of the active electronic components, the processor 210 can actively select and control which energy source(s) are used to power a particular active electronic component. Further, in embodiments having multiple types of energy storage component(s) 220, the processor 210 can direct the energy storage component(s) 220 to transfer power between the components. For example, a supercapacitor can be charged by the energy receiving component(s) 224 during a relatively short exposure, then the supercapacitor can charge a secondary battery over a longer period of time. Further details regarding suitable power management schedules for the system 200 may be found International Patent App. No. PCT/US2021/028931, which was previously incorporated herein by reference.

FIGS. 2B and 2C are schematic illustrations of the system 200 for wirelessly delivering energy to an implanted device (e.g., the shunting element 202) in accordance with embodiments of the present technology. In the illustrated embodiment, the system 200 is deployed in a space 240, such as the patient P's home bedroom, living room, kitchen, house, apartment, a hotel room, a clinical room, and/or any other suitable space. A remote device 218 is connected to and/or includes a power supply, such as the illustrated wall outlet, a battery pack, and/or any other suitable power supply. The remote device 218 is positioned to wirelessly communicate with (indicated by dashed lines) the shunting element 202. For example, as discussed above, the shunting element 202 can transfer sensor data to the remote device 218 and/or the remote device 218 can communicate instructions to the shunting element 202. Similarly, an energy transmission device 222 is connected to power supply 226 and positioned to wirelessly communicate with and transfer energy to (indicated by a solid line) the shunting element 202. In some embodiments, the power supply 226 is a small component that can be placed in a discreet location (e.g., underneath a bed or another piece of furniture). In such embodiments, the power supply 226 can include a power cord and an output cable for connection to a transmission coil system, a wireless energy transfer means to the transmission coil, and/or other energy transfer means. In some embodiments, the remote device 218 and the energy transmission device 222 can share a single power supply (e.g., power supply 226, a single battery pack, etc.).

As best seen in FIG. 2C, the energy transmission device 222 can be a structure (e.g., a structure containing one or more circuit boards) positioned beneath a mattress 252 that includes four transmission coils 228. When the patient P lays on the mattress 252, the four transmission coils 228 can be activated sequentially and/or in combination to generate varying electromagnetic fields. As a result, the energy transmission device 222 can effectively transfer energy to the shunting element 202, despite the potentially frequently changing and/or widely varying orientations of the shunting element 202 relative to the transmission device 222 while the patient P is on the mattress 252.

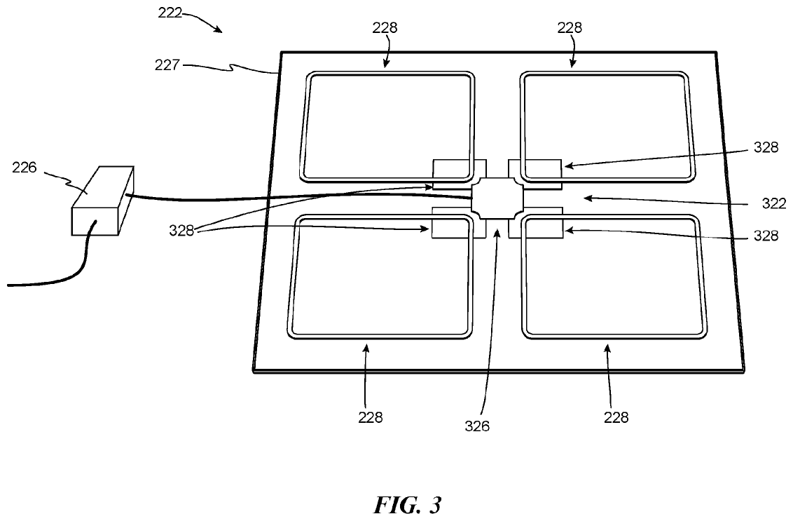

FIG. 3 is a partially schematic top view of the energy transmission device 222 for wirelessly delivering energy to an implanted device in accordance with some embodiments of the present technology. In the illustrated embodiment, the energy transmission device 222 includes a housing 227, a plurality of transmission coils 228 positioned within the housing 227, and a controller system 322 operably coupled to the transmission coils 228. The controller system 322 includes a controller 326 that is operably connected to a power supply 226, such as a wall-connected power supply, and a plurality of drivers 328 operably connected to the controller 326. Each individual driver 328 is connected to a corresponding individual transmission coil 228. Accordingly, the controller 326 can draw power from the power supply 226 and instruct the drivers 328 to energize the transmission coils 228 sequentially and/or in any suitable combination to generate varying electromagnetic fields. In other embodiments, a single driver 328 can be connected to more than one transmission coil 228, and electronic circuitry and/or software within the controller system 322 can determine which coil(s) 228 are energized by the driver 328 at any given time.

In various embodiments, the energy transmission device 222 can include any other suitable number of transmission coils 228. For example, although the energy transmission device 222 in the illustrated embodiment comprises four coils, in other embodiments the energy transmission device 222 can include one, two, five, ten, or any other suitable number of transmission coils. In some embodiments, the transmission coils 228 are all of similar size and shape. In other embodiments, however, the transmission coils may vary in size and/or shape. Further, in various embodiments, the transmission coils 228 can be arranged in any suitable distribution. For example, in the illustrated embodiment, the transmission coils 228 are arranged in a grid. In various other embodiments, the transmission coils 228 (or some subset thereof) can be spaced non-uniformly relative to other transmission coils 228. In some embodiments, some transmission coils 228 are arranged concentric with other transmission coils 228. In some embodiments, some transmission coils 228 vary in position relative to other transmission coils 228 in more than one spatial plane. Accordingly, as one skilled in the art will recognize, any number or spatial combination of coil circuits is possible.

The use of the higher radiofrequencies discussed above can affect the way currents travel along conductors that form the transmission coils 228. For example, without wishing to be bound by theory, it is believed that at relatively higher frequencies, only the outer portion (e.g., the portion near a perimeter or exterior) of a conductor carries meaningful current. Consequently, in some embodiments, the transmission coils 228 have a tubular conductor profile that surrounds a non-conductive inner portion (e.g., fiberglass), rather than a solid conductive profile. In some embodiments, the conductor profile can be achieved using Litz wire (e.g., constructed with a large number of individually insulated small wire strands) and/or with conductive tubes (e.g., copper tubes). However, a portion of a patient's weight transferred to the structure of the transmission coils 228 during use (e.g., when the energy transmission device 222 is integrated into or underneath a mattress, seat cushion, chair liner, or rug) can damage the Litz wire or tubular configurations. Further, the Litz wire or tubular configurations can be perceived as physically uncomfortable for the patient to interface with (e.g., lay on). Accordingly, as discussed in more detail below, in some embodiments the transmission coils 228 may be constructed within a flat structure containing a printed circuit board or a plurality of printed circuit boards. The flat structure can be strong enough to withstand the patient's weight and can be perceived as relatively more comfortable to the patient based on the flat upper surface while maintaining a tubular shape for the transmission coils 228. In some embodiments the flat structure is a flexible printed circuit board.

In some embodiments, utilizing the tubular conductive structure with a non-conductive interior for the transmission coils 228 is also expected to reduce the energy/utility power consumption of the energy transmission device 222. This can be at least in part due to the shape of the tubular conductors comprising such transmission coils 228, which can be relatively wide, thereby increasing surface area of the conductor and reducing losses related to electrical resistance. Further, the expected reduction in the energy/utility power consumption of the energy transmission device 222 from the tubular conductive structure for the transmission coils 228 does not require large masses of metallic material, thereby avoiding extraneous weight and/or associated costs. Power consumption may be further reduced by employing switch-mode power topologies for all power conversions from an initial power source (e.g., wall outlet power) to the radiofrequency drive power. Further, some embodiments can utilize extremely low-loss electronic components within the controller system 322, such as inductors, capacitors, and MOSFETs, that are rated for operation at substantially higher powers than the operation of the system 200 requires. Any of the power consumption reduction schemes discussed above can reduce the financial operating cost of the energy transmission device 222 and can be used in any combination. Further all of the reductions in the financial operating cost of the energy transmission device 222 are expected to improve patient compliance with an intended use of the energy transmission device 222 relative to charging systems described in the prior art.

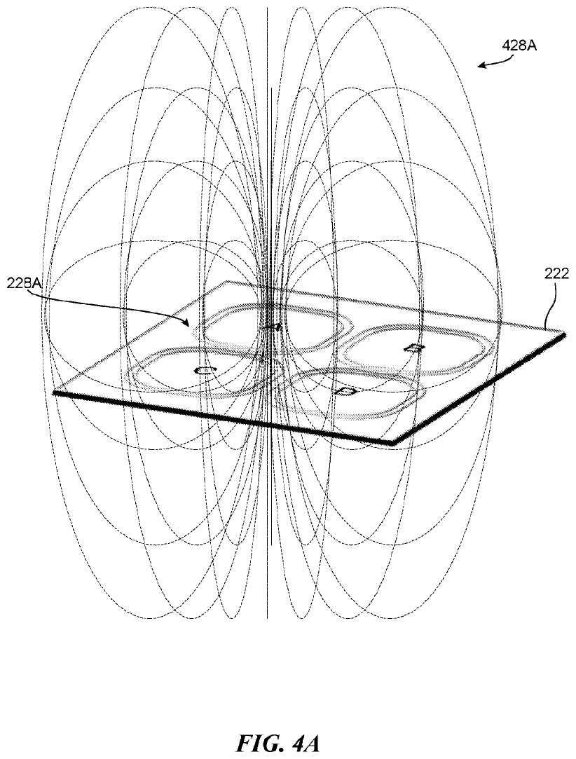

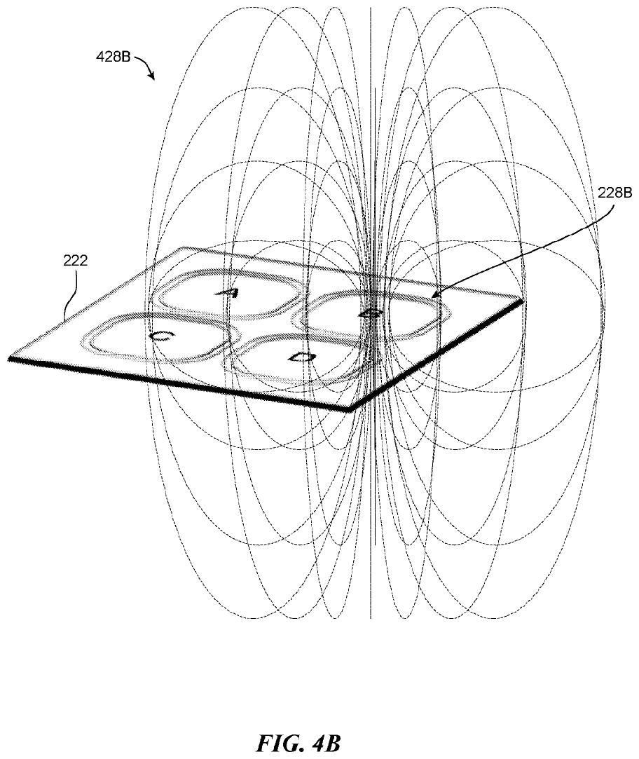

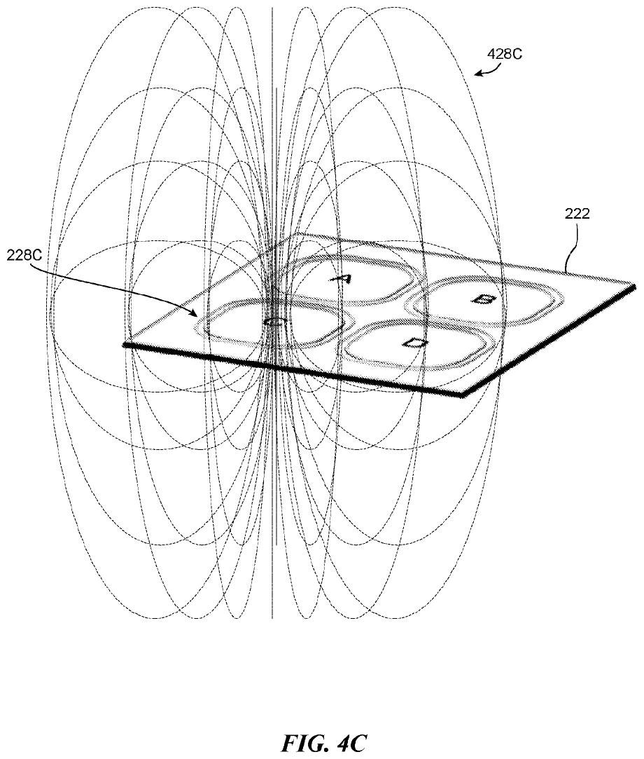

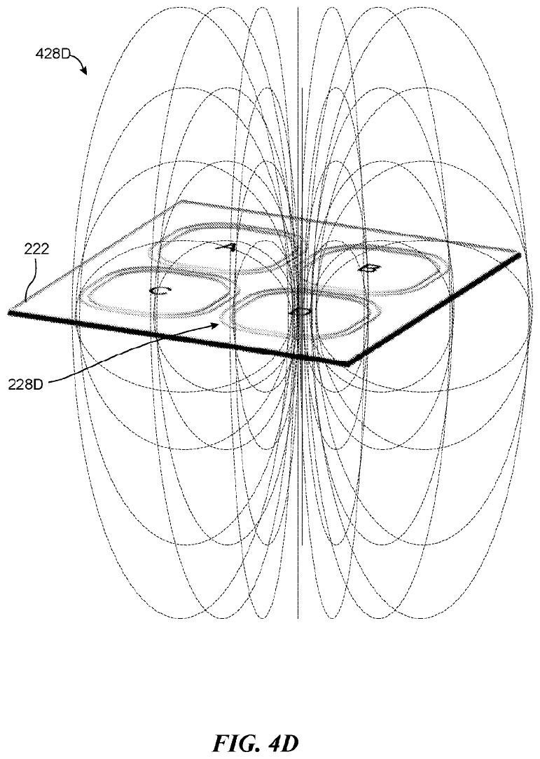

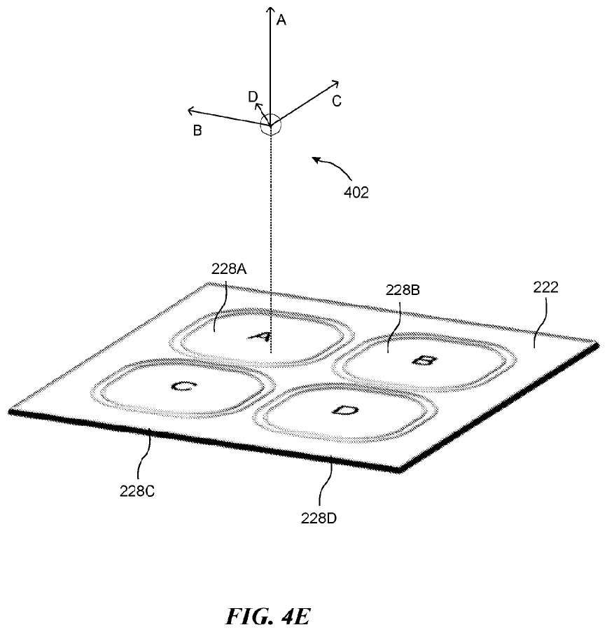

FIGS. 4A-4D are schematic diagrams of the magnetic fields produced by the energy transmission device 222 having four transmission coils 228 (referred to individually as the transmission coils 228A-D) in accordance with some embodiments of the present technology. In the illustrated embodiment, the transmission coils 228 are energized individually in sequence, starting with the transmission coil 228A (FIG. 4A) and ending with the transmission coil 228D (FIG. 4D). As a result, the transmission coils 228A-D create four independent magnetic fields 428A-D. As illustrated, the magnetic field 428A (FIG. 4A) has a closed loop shape that runs through the center of the transmission coil 228A; the magnetic field 428B (FIG. 4B) has a closed loop shape that runs through the center of the transmission coil 228B; the magnetic field 428C (FIG. 4C) has a closed loop shape that runs through the center of the transmission coil 228C; and the magnetic field 428D (FIG. 4D) has a closed loop shape that runs through the center of the transmission coil 228D. At the location of an implanted device positioned above the energy transmission device 222, the magnetic fields 428A-D can have differing orientations and/or field magnitudes. In some embodiments, a variation in the field intensity can be within a predetermined ratio for a given location, such as a variation of no more than about 2.5:1 over a 2 m×2 m area centered about 1 m above the charging mat.

FIG. 4E is a schematic vector diagram of the magnetic field components at the location of an implanted device 402 positioned above the energy transmission device 222 in accordance with some embodiments of the present technology. In the illustrated embodiment, the implanted device 402 is represented schematically as a point particle located vertically above the center of the transmission coil 228A. In this position, the implanted device 402 can interact with components from each of the magnetic fields 428A-D (FIGS. 4A-4D), represented by vector arrows A-D. As illustrated in FIG. 4E, the vector arrows A-D of the magnetic field at the location of the implanted device 402 can have components in multiple orientations. For example, vector arrow A has only components in a vertical direction; vector arrow B has components in a first horizontal direction and the vertical direction; vector arrow C has components in a second horizontal direction different from the first horizontal direction and the vertical direction; and vector arrow D has components in a third horizontal direction different from the first and second horizontal directions and the vertical direction. In some embodiments, any combination of the transmission coils 228A-D can additionally, or alternatively, be energized concurrently, resulting in interaction between the generated magnetic fields (not shown) to form further magnetic fields and therefore further magnetic field components with further orientations at the location of the implanted device 402.

As a result of the multiple magnetic field orientations produced by the energy transmission device 222, the receiving coil(s) in the implanted device 402 will be at least partially aligned with at least one of the components from the magnetic fields 428A-D in most patient positions (relative to the energy transmission device 222). Accordingly, the implanted device 402 will receive at least a portion of the energy transmitted by the energy transmission device 222 as the energy transmission device 222 sequentially energizes the transmission coils 228A-D. As discussed above, the transmission coils 228A-D can be energized cyclically for a predetermined time period, resulting in the transmission of the magnetic fields 428A-D cyclically for the predetermined time period. The energy transmission device 222 can deliver energy to the implanted device 402 during the periods in which the receiving coil(s) are at least partially aligned with one of the magnetic fields 428A-D. Accordingly, if the implanted device 402 is near the energy transmission device 222 for some extended period of time (e.g., when the patient is sleeping and the energy transmission device 222 is integrated into or otherwise proximate to the patient's mattress), the energy transmission device 222 can effectively transfer energy to the implanted device 402 without the need for precise alignment between the transmission coils and the receiving coil(s). In some embodiments, for example, the energy received by the energy receiving component(s) of the implant 402 can be about 100 μW to about 2 mW, depending on, among other things, the patient's weight, patient position(s), and the bed construction. Likewise, the charging time can vary from about 15 minutes to about 4 hours depending upon these same variables.

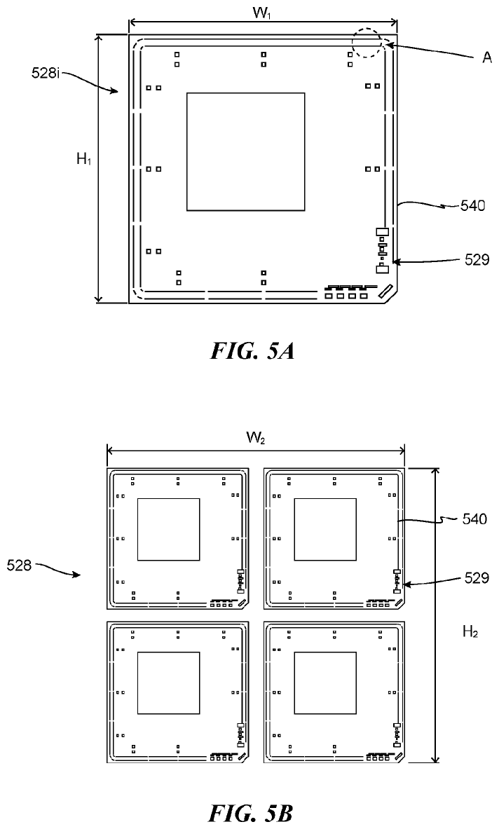

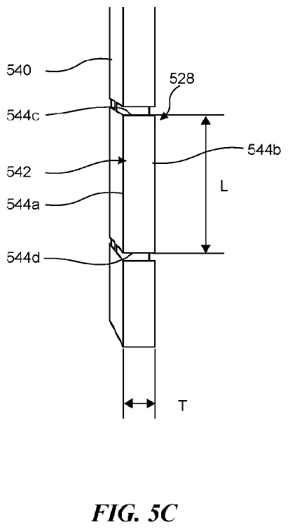

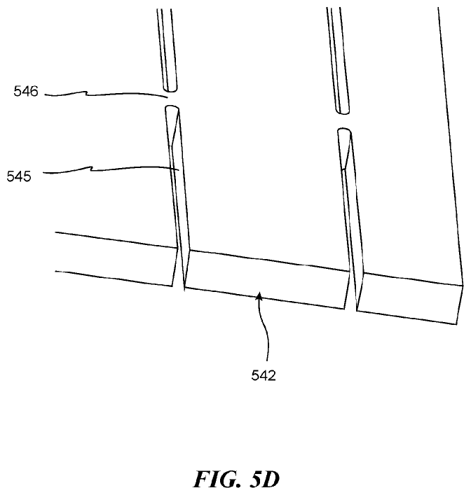

FIGS. 5A and 5B are partially schematic top views of transmission coils 528 and corresponding drivers 529 (only one labeled in FIG. 5B) manufactured on a printed circuit board 540 in accordance with some embodiments of the present technology. The drivers 529 can receive instructions from a controller (e.g., controller 326 discussed above with respect to FIG. 3) to drive a current through the transmission coils 528 (sometimes also referred to as “energizing”), thereby generating a magnetic field to transfer energy to an implanted device. Accordingly, the printed circuit board 540) can be incorporated into and/or comprise the energy transmission device 222 (e.g., FIGS. 2A and 3), and can improve the load bearing capacity of and/or the comfort of using the energy transmission device 222. The increase in durability is especially useful in embodiments in which the patient's weight is at least partially conveyed to the energy transmission device 222 (e.g., when the energy transmission device 222 is positioned beneath a mattress).

In some embodiments, the transmitting coils 528 manufactured on the printed circuit board 540) are designed to maximize the cross-sectional area of a conductive material (e.g., copper, silver, silver-plated copper, etc.) available to conduct a desired radiofrequency current. Further, in some embodiments, the transmission coils 528 have a relatively large overall footprint. For example, FIG. 5A illustrates an individual transmission coil 528i that has a width W₁ and a height H₁. Each of the width W₁ and the height H₁ can be between about 4 inches (in.) to about 25 in. In the illustrated embodiment, the individual transmission coil 528i has a rectangular shape with the width W₁ representing one side and the height Hi representing another side. In some embodiments, the individual transmission coil 528i has a continuous circular shape with a radius represented by either the width W₁ or the height H₁ and surrounded by a rectangular frame that that adds between about 1 in. and about 10 in. to the printed circuit board (or section thereof) containing the individual transmission coil 528i. In various other embodiments, the individual transmission coil 528i can have various other shapes (e.g., a hexagon, an octagon, an elliptical shape, or any other suitable shape) with a footprint defined by the width W₁ and the height H₁. As illustrated in FIG. 5B, the printed circuit board 540 comprising multiple transmission coils 528 has a width W₂ and a height H₂ corresponding to the sum of the footprints of the transmission coils 528 and any buffer space between the transmission coils 528. For example, in the illustrated embodiment, the width W₂ and the height H₂ can each be between about 15 in. to about 60 in. depending on the size of the transmission coils 528 and corresponding buffer space. In other embodiments, the printed circuit board 540 may have other dimensions/shapes.

FIG. 5C and FIG. 5D are partially schematic cross-sectional views of an individual transmitting coil 528 manufactured on a printed circuit board 540 in accordance with some embodiments of the present technology. The printed circuit board 540 can be a traditional printed circuit board material comprised of a laminated composite material, a flex-type circuit board, a thin FR4 circuit board, and/or can be comprised of other suitable materials. During manufacturing, one or more tubular structures 542 (also referred to as “the tubular structure 542”) can be created on the printed circuit board 540 by connecting structures on upper and lower surfaces 544a, 544b of the printed circuit board 540) with conductive structures 544c, 544d located on the surfaces of one or more elongated slots 545 therebetween. That is, in some embodiments, the tubular structure 542 is comprised of upper and lower conductive surfaces connected by additional conductive surfaces on the walls of elongated slots 545 in the circuit board 540). In some embodiments, the elongated slots 545 in the printed circuit board 540 include periodic interruptions 546 (FIG. 5D) that help maintain the physical integrity of the printed circuit board 540) and the tubular structure 542. When the interruptions 546 are small relative to the slots, they will not significantly affect the electrical performance of the transmission coils 528. In various embodiments, the elongated slots 545 can be 1 in. long, 3 in. long, 5 in. long, and/or more than 5 in. long. In some embodiments, the top, bottom, and slotted sections of the conductive structures that form the coil can all use the same conductive material, or the conductive material can vary depending on the region of the coil. In the illustrated embodiment, the conductive structures and elongated slots 545 define a single tubular structure 542 with a rectangular cross section. In other embodiments, however, the conductive structures and/or elongated slots 545 may have a different configuration.

In some embodiments, the transmission coils 528 can be include relatively thin conductive structures. For example, the transmission coils 528 can have a thickness T of between about 0.001 in. and about 0.005 in. In some embodiments of the system 200 (FIG. 2A) utilizing a lower frequency than 6.78 MHz, the thickness of the conductive material in the transmission coils 528 are scaled thicker by at least a ratio of √(6.78 MHz/f). In various embodiments, the thickness of the conductive structure in the conductive slots 545 can be identical to, thicker than, or thinner than the thickness of the conductive structures on the top and bottom regions of the circuit board. As further illustrated in FIG. 5C, each of the transmission coils 528 (one shown) has a relatively wide width L. For example, the width L can be between about 0.25 in., about 0.5 in., about 1 in., or more than 1 in. In some embodiments, the width L can be generally homogenous around the transmission coils 528. In other embodiments, however, the width L can vary according to a position around the transmission coils 528.

As discussed above, forming transmission coils 528 on the flat printed circuit board 540 is desirable to incorporate the transmission coils 528 into a surface that the patient routinely interfaces with (e.g., a mat under a mattress, a seat cushion, a rug, etc.). For example, the flattened nature of the design reduces the visual presence of the energy transmission device 222 (see, e.g., FIG. 2B) and allows for a smooth surface that a patient may consider more comfortable to interact with (e.g., a patient laying on top of a smoother surface). Further, forming the transmission coils 528 on the flat printed circuit board 540 is expected to increase the structural integrity of the energy transmission device 222, especially in embodiments in which the energy transmission device 222 is exposed to any of the patient's weight.

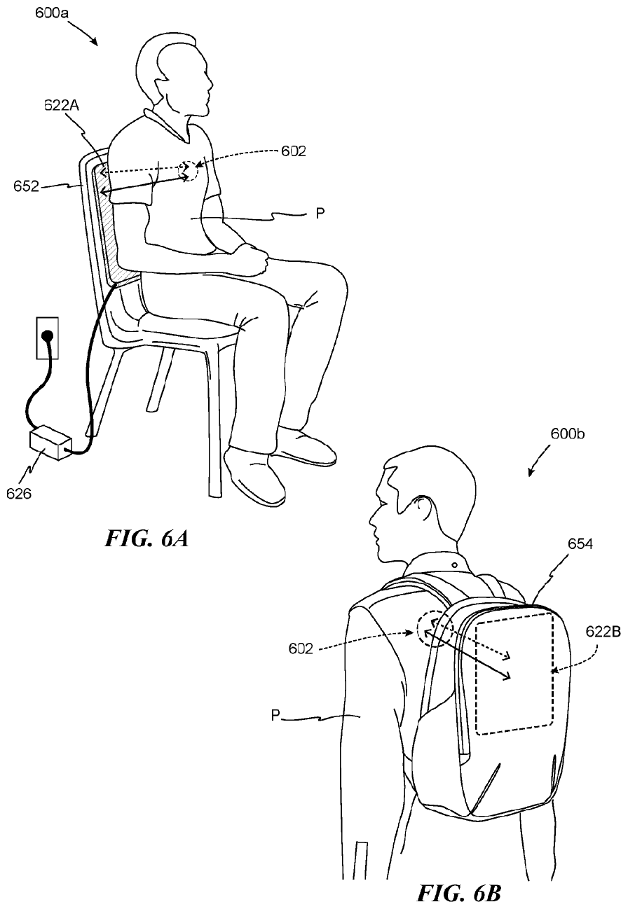

FIGS. 6A and 6B are schematic illustrations of systems 600a, 600b (respectively) of the type illustrated in FIG. 2A for wirelessly delivering energy configured in accordance with further embodiments of the present technology. For example, as illustrated with respect to FIG. 6A, the system 600a includes an implanted device 602 (e.g., the shunting element 202 of FIG. 2A) positioned within a patient P's body and an energy transmission device 622a that emits electromagnetic fields to communicate with and/or wirelessly deliver energy to the implanted device 602. The energy transmission device 622a is connected to a power supply 626. In the illustrated embodiment, the energy transmission device 622a is integrated into a seating element 652 (e.g., a chair, desk chair, stool, couch, or another suitable element) that the patient P interacts with on a recurring basis (e.g., hourly, daily, weekly, etc.). In one embodiment, for example, the energy transmission device 622a is incorporated into the patient P's office desk chair to predictably deliver energy to the implanted device 602 while the patient P is at work. In the illustrated embodiment, the power supply 626 is in turn connected to a wall outlet to draw power from the wall outlet. In some embodiments, the power supply 626 can be a primary and/or secondary battery cell, allowing the energy transmission device 622a to be mobile between various seating elements 652.

The energy transmission device 622a integrated into the seating element 652 can include multiple coils that are energized sequentially in the manner discussed above. Through the sequential energization, the energy transmission device 622a can transfer energy to the implanted device 602 without requiring careful alignment and/or positioning of the patient P. As a result, the patient P can engage with the seating element 652 without additional consideration to their position and still receive an adequate energy transfer from the energy transmission device 622a.

In the embodiment illustrated in FIG. 6B, the system 600b includes an energy transmission device 622b integrated into a portable unit 654 to communicate with and/or wirelessly deliver energy to the implanted device 602. In various embodiments, the energy transmission device 622b can be integrated into a blanket, jacket, backpack, and/or any other suitable unit. As integrated into the portable unit 654, the energy transmission device 622b can still include multiple coils that are energized sequentially in the manner discussed above. Through the sequential energization, the energy transmission device 622b can transfer energy to the implanted device 602 without careful alignment and/or positioning of the portable unit 654. As a result, the patient P can engage with the portable unit 654 without carefully aligning and/or positioning the portable unit 654 and still receive an adequate energy transfer from the energy transmission device 622b.

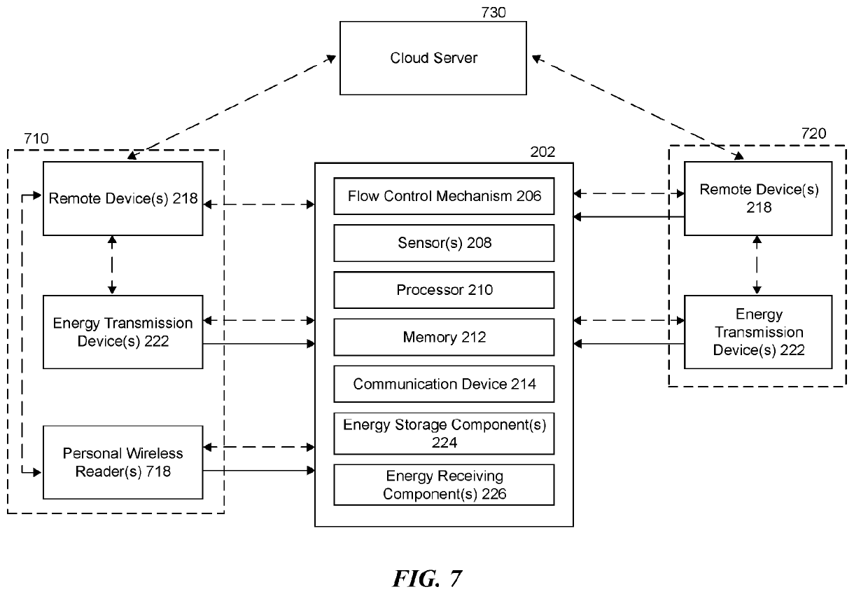

FIG. 7 is a block diagram of a system 700 for wirelessly delivering energy to a shunting element 202 configured in accordance with further embodiments of the present technology. As illustrated, the system 700 is generally similar to the system 200 discussed above with respect to FIG. 2A. For example, the shunting element 202 includes a flow control mechanism 206, one or more sensor(s) 208, a processor 210, memory 212, a communication device 214, one or more energy storage components 220, and one or more energy receiving component(s) 224 operably coupled to the shunting element 202 and/or components thereof.

In the illustrated embodiment, the system 700 also includes a home subsystem 710 and a clinical subsystem 720 that each contain components to communicate with and/or transfer energy to the shunting element 202. For example, the home subsystem 710 includes one or more remote devices 218, one or more energy transfer devices 222, and one or more personal wireless readers 718. As discussed in detail above, the remote device(s) 218 can communicate with the shunting element 202 (e.g., through the communication device 214) and the energy transfer device(s) 222 can communicate with and/or transfer energy to the shunting element 202 (e.g., through the communication device 214 and/or the energy receiving component(s) 224). As further illustrated in FIG. 7, the energy receiving component(s) 224 can also wirelessly communicate with the remote device(s) 218. For example, the energy receiving component(s) 224 can prompt the remote device(s) 218 when they detect the presence of the shunting element 202 (e.g., using the presence sensors discussed above) and/or can relay data from the shunting element 202.

In another example, the remote device(s) 218 can periodically communicate with (e.g., by sending and/or receiving test signals) the energy transfer device(s) 222, through which the remote device(s) 218 can interrogate the current power status of the energy transfer device(s) 222. If the remote device(s) 218 determine that the energy transfer device(s) 222 have become non-operational (e.g., when the remote device(s) 218 do not receive a scheduled/anticipated test signal or return signal), the remote device(s) 218 can alert the patient, a care provider, or another suitable party to the status of the energy transfer device(s) 222. The periodic queries are expected to help prevent extended periods of non-operation of the energy transfer device(s) 222, for example those due to disconnection from a power source or those due to malfunction. In some embodiments, the remote device(s) 218 have additional functionalities. For example, the remote device(s) 218 can serve as a data reader that can extract, store, interpret, process, or otherwise interface with patient data that is captured by or stored on the shunting element 202 and/or the energy transfer device(s) 222.

In the illustrated embodiment, the home subsystem 710 also includes a personal wireless reader 718 that can communicate with the shunting element 202 in a similar manner as described above with respect to the remote device(s) 218. In some embodiments, the personal wireless reader 718 can be a body worn device (e.g., a smart watch or other suitable device) and/or any other portable device the patient is likely to carry with them. In such embodiments, the shunting element 202 can communicate alerts to the personal wireless reader 718 to be relayed to the patient. For example, the alerts can include indications that the energy storage components 220 are running low, indications that one or more components of the shunting element 202 are malfunctioning, sensor data indicating a current patient status measured by the sensors, and/or any other suitable alert.

Further, in the illustrated embodiment, the home subsystem 710 can communicate with a cloud server 730) (e.g., over a network connection). In some embodiments, the home subsystem 710 communicates with the cloud server 730 to upload data received from the shunting element 202, metrics on the activity of the energy receiving component(s) 224, metrics on the energy storage components 220 and/or the consumption of the shunting element 202, and/or any other suitable data to a cloud storage device. The data can then be reviewed by the patient and/or a care provider on another device connected to the cloud server 730 (e.g., the patient's personal computer, the patient's smartphone, and/or a computer at a care center, and/or any other networked device). In some embodiments, the cloud server 730 communicates with the home subsystem 710 to provide instructions that can be relayed to the shunting element 202, to provide instructions to prompt the shunting element 202 for specific data, to relay a message to the patient, and/or for any other suitable purpose. For example, a care provider reviewing the data uploaded to the cloud server 730 may determine that the shunting element 202 should make one or more adjustments and can relay instructions corresponding to the one or more adjustments to the shunting element 202 through the cloud server 730 and the home subsystem 710.

The clinical subsystem 720 can also include one or more remote devices 218 and one or more energy transfer devices 222. Accordingly, the clinical subsystem 720 is generally similar in function to the home subsystem 710 discussed above. However, the embodiments of the remote device(s) 218 and/or the energy transfer device(s) 222 can vary between the clinical subsystem 720 and the home subsystem 710. For example, the energy transfer device(s) 222 in the clinical subsystem 720 can be configured to deliver more energy per minute (e.g., higher power) to the shunting element 202 in the controlled clinical environment. Similarly, the remote device(s) 218 in the clinical subsystem 720 may vary from the remote device(s) 218 in the home subsystem 710 both in structure and in the data the remote device(s) 218 prompt the shunting element 202 to communicate. For example, the remote device(s) 218 can receive instructions from a care provider to specifically prompt the shunting element 202.

In the illustrated embodiment, the clinical subsystem 720 can also communicate with a cloud server 730 (e.g., over the network connection). Similar to the discussion of the home subsystem 710 above, in some embodiments, the clinical subsystem 720 communicates with the cloud server 730 to upload data received from the shunting element 202, metrics on the activity of the energy receiving component(s) 224, metrics on the energy storage components 220 and/or the consumption of the shunting element 202, and/or any other suitable data to a cloud storage device. The data can then be reviewed by a local care provider on another device connected to the cloud server 730 and/or another care provider remote to the clinical subsystem 720. Further, in some embodiments, the cloud server 730 communicates with the clinical subsystem 720 for any of the functions discussed above. For example, a remote care provider reviewing the data uploaded to the cloud server 730 may determine that the shunting element 202 should make one or more adjustments and can relay instructions corresponding to the one or more adjustments to the shunting element 202 through the cloud server 730 and the clinical subsystem 720.

### C. Examples

Several aspects of the present technology are set forth in the following examples:

1. A system for wirelessly transferring energy to an implanted device positioned in a patient's body, the system comprising:
an active electronic component carried by the implanted device in the patient's body;
one or more energy storage components operably coupled to the implanted device, wherein at least one of the one or more energy storage components is rechargeable;
at least one receiving coil operably connected to at least one of the one or more energy storage components; and
an energy transmission device external to the patient, wherein the energy transmission device comprises a plurality of transmission coils at least partially offset from each other in a longitudinal plane,
wherein, during operation, each of the transmission coils transmits a magnetic field, and
wherein, when the at least one receiving coil is at least partially aligned with the magnetic field and within a predetermined range of the energy transmission device, the magnetic field induces a current in the at least one receiving coil to deliver energy to the at least one receiving coil, wherein the received energy is stored in the one or more energy storage components for powering the active electronic component.

2. The system of example 1, further comprising a presence sensor operably coupled to the energy transmission device and positioned to detect a presence of the implanted device within the predetermined range of the energy transmission device, and wherein the energy transmission device is configured to operate in response to the detected presence.

3. The system of example 1 or example 2 wherein the energy transmission device further includes a controller, the controller including:
a processor; and
a memory storing instructions that, when executed by the processor, cause the controller to perform operations comprising—
   energizing a first transmission coil from the plurality of transmission coils to generate a first magnetic field; and
   energizing a second transmission coil from the plurality of transmission coils to generate a second magnetic field at least partially offset from the first magnetic field.

4. The system of example 3 wherein when the at least one receiving coil is at least partially aligned with the first magnetic field within the predetermined range, the first magnetic field induces a first current in the at least one receiving coil.

5. The system of example 3 wherein when the at least one receiving coil is at least partially aligned with the second magnetic field within the predetermined range, the second magnetic field induces a second current in the at least one receiving coil.

6. The system of any one of examples 3-5 wherein the instructions further cause the controller to perform operations comprising:
energizing a third transmission coil from the plurality of transmission coils to generate a third magnetic field at least partially offset from the first and second magnetic fields; and
energizing a fourth transmission coil from the plurality of transmission coils to generate a fourth magnetic field at least partially offset from the first, second, and third magnetic fields.

7. The system of example 6 wherein, when the at least one receiving coil is at least partially aligned with the third magnetic field within the predetermined range, the third magnetic field induces a third current in the at least one receiving coil, and wherein when the at least one receiving coil is at least partially aligned with the fourth magnetic field within the predetermined range, the fourth magnetic field induces a fourth current in the at least one receiving coil.

8. The system of any one of examples 3-7 wherein the instructions further cause the controller to perform operations comprising energizing the first and second transmission coils to generate a fifth magnetic field at least partially offset from the first and second magnetic fields.

9 The system of example 8 wherein, when the at least one receiving coil is at least partially aligned with the fifth magnetic field within the predetermined range, the fifth magnetic field induces a fifth current in the at least one receiving coil.

10. The system of any one of examples 1-9 wherein the active electronic component is a communication device, and wherein the system further comprises a remote device configured to wirelessly communicate with the communication device.

11. The system of example 10, further comprising one or more sensors positioned to collect data on one or more physiological parameters of the patient, and wherein the communication device is operably coupled to the one or more sensors to communicate the data the one or more physiological parameters of the patient to the remote device.

12. The system of any one of examples 1-11 wherein the magnetic field has a transmission frequency of 6.8 MHz and/or 13.56 MHz.

13. The system of any one of examples 1-12 wherein the energy transmission device further includes a housing having a flat upper surface.

14. The system of example 13 wherein housing comprises a printed circuit board.

15. The system of any one of examples 1-15 wherein each of the plurality of transmission coils has a tubular construction.

16. The system of any one of examples 1-15, further comprising a cloud server operably coupled to at least one of the energy transmission device and the remote device.

17. The system of any one of examples 1-16 wherein at least one of the one or more energy storage components comprises a supercapacitor.

18. A method for wirelessly transferring energy to an implanted device positioned within a patient's body, the method comprising:
energizing a first transmission coil, wherein energizing the first transmission coil causes the first transmission coil to transmit a first magnetic field having a first spatial orientation; and
energizing a second transmission coil at least partially offset from the first transmission coil, wherein energizing the second transmission coil causes the second transmission coil to transmit a second magnetic field having a second spatial orientation at least partially offset from the first spatial orientation.

19. The method of example 18 wherein the first magnetic field transfers energy to a receiving coil operably coupled to the implanted device when the receiving coil is at least partially aligned with the first magnetic field in the first spatial orientation, and wherein the second magnetic field transfers energy to the receiving coil operably coupled to the implanted device when the receiving coil is at least partially aligned with the second magnetic field in the second spatial orientation.

20. The method of example 18 or example 19 wherein the first and/or second magnetic fields can be used to transfer energy to a supercapacitor operably coupled to the implanted device.

21. The method of any one of examples 18-20, further comprising detecting a presence of an implanted device within a predetermined range of an energy transmission device housing at least one of the first and second transmission coils.

22. The method of any one of examples 18-21, further comprising:
energizing a third transmission coil at least partially offset from the first and second transmission coils, wherein energizing the third transmission coil causes the third transmission coil to transmit a third magnetic field having a third spatial orientation at least partially offset from the first and second spatial orientations; and
energizing a fourth transmission coil at least partially offset from the first, second, and third transmission coils, wherein energizing the fourth transmission coil causes the fourth transmission coil to transmit a fourth magnetic field having a fourth spatial orientation at least partially offset from the first, second, and third spatial orientations.

23. The method of example 22 wherein the third magnetic field transfers energy to the receiving coil operably coupled to the implanted device when the receiving coil is at least partially aligned with the third magnetic field in the third spatial orientation, and wherein the fourth magnetic field transfers energy to the receiving coil operably coupled to the implanted device when the receiving coil is at least partially aligned with the fourth magnetic field in the fourth spatial orientation.

24. The method of example 22 wherein energizing of the first, second, third, and fourth transmission coils occurs in a cyclical process.

25. The method of any one of examples 18-24, further comprising energizing the first and second transmission coils together, wherein energizing the first and second transmission coils causes the first and second transmission coils to transmit interacting magnetic fields that create a fifth magnetic field having a fifth spatial orientation at least partially offset from the first and second spatial orientations, and wherein the fifth magnetic field transfers energy to the receiving coil operably coupled to the implanted device when the receiving coil is at least partially aligned with the fifth magnetic field in the fifth spatial orientation.

26. The method of any one of examples 18-25 wherein the energizing the first transmission coil causes the first transmission coil to generate the first magnetic field with a transmission frequency of 6.78 MHz and/or 13.56 MHz.

27. The method of any one of examples 18-26 wherein the implanted device includes a communication device and a sensor positioned to measure at least one physiological parameter of the patient, and wherein the communication device is operably connected to the sensor, and further wherein the method further comprises wirelessly receiving, from the communication device, data related the at least one physiological parameter of the patient.

28. The method of example 27, further comprising communicating, to a cloud server, the data related to the at least one parameter of the patient's body.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; non-programmable components (e.g., diodes, comparators, gates, MOSFETS, etc.) that drive operation of an implant; memory such as RAM or ROM to store data and/or software/firmware associated with an implant and/or its operation; wireless communication hardware such as an antenna system configured to transmit via Bluetooth, WiFi, LoRa, Thread, Zigbee, UWB, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

Embodiments of the present disclosure may be implemented as computer-executable instructions, such as routines executed by a general-purpose computer, a personal computer, a server, or other computing system. The present technology can also be embodied in a special purpose computer or data processor that is specifically programmed, configured, or constructed to perform one or more of the computer-executable instructions explained in detail herein. The terms “computer” and “computing device,” as used generally herein, refer to devices that have a processor and non-transitory memory, as well as any data processor or any device capable of communicating with a network. Data processors include programmable general-purpose or special-purpose microprocessors, programmable controllers, ASICs, programming logic devices (PLDs), or the like, or a combination of such devices. Computer-executable instructions may be stored in memory, such as RAM, ROM, flash memory, or the like, or a combination of such components. Computer-executable instructions may also be stored in one or more storage devices, such as magnetic or optical-based disks, flash memory devices, or any other type of non-volatile storage medium or non-transitory medium for data. Computer-executable instructions may include one or more program modules, which include routines, programs, objects, components, data structures, and so on that perform particular tasks or implement particular abstract data types.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the LA and RA, the LV and the right ventricle (RV), or the LA and the coronary sinus, it should be appreciated that similar embodiments could be utilized for shunts between other chambers of heart or for shunts in other regions of the body.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for wirelessly transferring energy to an implanted device positioned in a patient's body, the system comprising:
an active electronic component carried by the implanted device in the patient's body;
one or more energy storage components operably coupled to the implanted device, wherein at least one of the one or more energy storage components is rechargeable;
at least one receiving coil operably connected to at least one of the one or more energy storage components; and
an energy transmission device external to the patient, wherein the energy transmission device comprises a plurality of transmission coils at least partially offset from each other in a longitudinal plane,
wherein, during operation, each of the transmission coils transmits a magnetic field, and
wherein, when the at least one receiving coil is at least partially aligned with the magnetic field and within a predetermined range of the energy transmission device, the magnetic field induces a current in the at least one receiving coil to deliver energy to the at least one receiving coil, wherein the received energy is stored in the one or more energy storage components for powering the active electronic component.

2. The system of claim 1, further comprising a presence sensor operably coupled to the energy transmission device and positioned to detect a presence of the implanted device within the predetermined range of the energy transmission device, and wherein the energy transmission device is configured to operate in response to the detected presence.

3. The system of claim 1 wherein the energy transmission device further includes a controller, the controller including:
a processor; and
a memory storing instructions that, when executed by the processor, cause the controller to perform operations comprising—
energizing a first transmission coil from the plurality of transmission coils to generate a first magnetic field; and
energizing a second transmission coil from the plurality of transmission coils to generate a second magnetic field at least partially offset from the first magnetic field.

4. The system of claim 3 wherein when the at least one receiving coil is at least partially aligned with the first magnetic field within the predetermined range, the first magnetic field induces a first current in the at least one receiving coil.

5. The system of claim 3 wherein when the at least one receiving coil is at least partially aligned with the second magnetic field within the predetermined range, the second magnetic field induces a second current in the at least one receiving coil.

6. The system of claim 3 wherein the instructions further cause the controller to perform operations comprising:
energizing a third transmission coil from the plurality of transmission coils to generate a third magnetic field at least partially offset from the first and second magnetic fields; and
energizing a fourth transmission coil from the plurality of transmission coils to generate a fourth magnetic field at least partially offset from the first, second, and third magnetic fields.

7. The system of claim 6 wherein, when the at least one receiving coil is at least partially aligned with the third magnetic field within the predetermined range, the third magnetic field induces a third current in the at least one receiving coil, and wherein when the at least one receiving coil is at least partially aligned with the fourth magnetic field within the predetermined range, the fourth magnetic field induces a fourth current in the at least one receiving coil.

8. The system of claim 3 wherein the instructions further cause the controller to perform operations comprising energizing the first and second transmission coils to generate a fifth magnetic field at least partially offset from the first and second magnetic fields.

9. The system of claim 8 wherein, when the at least one receiving coil is at least partially aligned with the fifth magnetic field within the predetermined range, the fifth magnetic field induces a fifth current in the at least one receiving coil.

10. The system of claim 1 wherein the active electronic component is a communication device, and wherein the system further comprises a remote device configured to wirelessly communicate with the communication device.

11. The system of claim 10, further comprising one or more sensors positioned to collect data on one or more physiological parameters of the patient, and wherein the communication device is operably coupled to the one or more sensors to communicate the data the one or more physiological parameters of the patient to the remote device.

12. The system of claim 1 wherein the magnetic field has a transmission frequency of 6.8 MHz and/or 13.56 MHz.

13. The system of claim 1 wherein the energy transmission device further includes a housing having a flat upper surface.

14. The system of claim 13 wherein housing comprises a printed circuit board.

15. The system of claim 1 wherein each of the plurality of transmission coils has a tubular construction.

16. The system of claim 1, further comprising a cloud server operably coupled to at least one of the energy transmission device and the remote device.

17. The system of claim 1 wherein at least one of the one or more energy storage components comprises a supercapacitor.

18. A method for wirelessly transferring energy to an implanted device positioned within a patient's body, the method comprising:
energizing a first transmission coil, wherein energizing the first transmission coil causes the first transmission coil to transmit a first magnetic field having a first spatial orientation; and
energizing a second transmission coil at least partially offset from the first transmission coil, wherein energizing the second transmission coil causes the second transmission coil to transmit a second magnetic field having a second spatial orientation at least partially offset from the first spatial orientation.

19. The method of claim 18 wherein the first magnetic field transfers energy to a receiving coil operably coupled to the implanted device when the receiving coil is at least partially aligned with the first magnetic field in the first spatial orientation, and wherein the second magnetic field transfers energy to the receiving coil operably coupled to the implanted device when the receiving coil is at least partially aligned with the second magnetic field in the second spatial orientation.

20. The method of claim 18 wherein the first and/or second magnetic fields can be used to transfer energy to a supercapacitor operably coupled to the implanted device.

21. The method of claim 18, further comprising detecting a presence of an implanted device within a predetermined range of an energy transmission device housing at least one of the first and second transmission coils.

22. The method of claim 18, further comprising:
energizing a third transmission coil at least partially offset from the first and second transmission coils, wherein energizing the third transmission coil causes the third transmission coil to transmit a third magnetic field having a third spatial orientation at least partially offset from the first and second spatial orientations; and
energizing a fourth transmission coil at least partially offset from the first, second, and third transmission coils, wherein energizing the fourth transmission coil causes the fourth transmission coil to transmit a fourth magnetic field having a fourth spatial orientation at least partially offset from the first, second, and third spatial orientations.

23. The method of claim 22 wherein the third magnetic field transfers energy to the receiving coil operably coupled to the implanted device when the receiving coil is at least partially aligned with the third magnetic field in the third spatial orientation, and wherein the fourth magnetic field transfers energy to the receiving coil operably coupled to the implanted device when the receiving coil is at least partially aligned with the fourth magnetic field in the fourth spatial orientation.

24. The method of claim 22 wherein energizing of the first, second, third, and fourth transmission coils occurs in a cyclical process.

25. The method of claim 18, further comprising energizing the first and second transmission coils together, wherein energizing the first and second transmission coils causes the first and second transmission coils to transmit interacting magnetic fields that create a fifth magnetic field having a fifth spatial orientation at least partially offset from the first and second spatial orientations, and wherein the fifth magnetic field transfers energy to the receiving coil operably coupled to the implanted device when the receiving coil is at least partially aligned with the fifth magnetic field in the fifth spatial orientation.

26. The method of claim 18 wherein the energizing the first transmission coil causes the first transmission coil to generate the first magnetic field with a transmission frequency of 6.78 MHz and/or 13.56 MHz.

27. The method of claim 18 wherein the implanted device includes a communication device and a sensor positioned to measure at least one physiological parameter of the patient, and wherein the communication device is operably connected to the sensor, and further wherein the method further comprises wirelessly receiving, from the communication device, data related the at least one physiological parameter of the patient.

28. The method of claim 27, further comprising communicating, to a cloud server, the data related to the at least one parameter of the patient's body.
